(19) United States
(12) Patent Application Publication
(10) Pub. No.: US 2023/0028307 A1
(43) Pub. Date: Jan. 26, 2023

(54) TREATMENT OF NEUROLOGICAL DISORDERS WITH AVERMECTINS

(71) Applicant: Equilibre BioPharmaceuticals BV, Amsterdam (NL)

(72) Inventors: Samuel D. Waksal, New York, NY (US); Ya-El Mandel-Portnoy, New York, NY (US); Rui Wu, Wilmington, DE (US)

(21) Appl. No.: 17/758,525

(22) PCT Filed: Jan. 11, 2021
(86) PCT No.: PCT/US21/12926

§ 371 Date: Jul. 8, 2022

Related U.S. Application Data
(60) Provisional application No. 62/959,629, filed on Jan. 10, 2020.

(51) Int. Cl. A61K 31/365; A61K 45/06

(52) U.S. Cl. CPC A61K 31/365; A61K 45/06

(57) ABSTRACT

Disclosed are methods of use of Ivermectin for the prevention, treatment and control of various neurological disorders in humans and novel formulations of pharmaceutical compositions comprising Ivermectin.

### FIELD OF THE INVENTION

The present invention relates to use of an avermectin compounds for the prevention, treatment and control of various neurological disorders in humans. Also disclosed are novel formulations for the administration of avermectins, and in particular ivermectin.

### BACKGROUND

The avermectins are a family of 16-membered macrocyclic lactone derivatives with potent anthelmintic and insecticidal properties and are used as active agents for the treatment or prevention of infection by parasitic worms and other parasitic infections. Avermectins are a series of macrolides, each of which is substituted thereon at the 13-position with a 4-(α-L-oleandrosyl)-α-L-oleandrose group. Avermectins are produced by cultures of the bacterium Streptomyces avermitilis or by synthetic or semi-synthetic means. The members of the avermectin family bind selectively and with high affinity to glutamate-gated chloride ion channels, which occur in invertebrate nerve and muscle cells. This leads to an increase in the permeability of the cell membrane to chloride ions with hyperpolarization of the nerve or muscle cell, resulting in paralysis and death of the parasite. All avermectin family of compounds show a similar spectrum of activity in different level of potency.

Ivermectin, an avermectin family member, is a highly potent anti-parasitic agent. Ivermectin is a mixture of 22, 23-dihydroavermectin B1a and 22, 23-dihydroavermectin B1b. Ivermectin has been used historically as a broad-spectrum anti-parasitic medicinal product for human and veterinary use.

Ivermectin is commercially available for animal use as Cardomec® (for felines), Eqvalane® (for equines) and Ivomec® (for bovines) by Merial; as Zimecterin® (for equines) by Famam Companies, Inc. The medicine is available in tablets and chewables for heartworm prevention, topical solution for ear mite treatment, and injectable solution, oral paste or solution for other parasites in veterinary use. Ivermectin is also available for human use for treating parasitic infestations. For example, Stromectol, marketed by Merck & Co. is approved by the U.S. Food and Drug Administration to treat onchoceriasis (river blindness) and strongyloidiasis (non-disseminated intestinal threadworm). Studies have noted that ivermectin caused no significant side effects even when administered in relatively large doses to the humans. Topical application of ivermectin have been described for the treatment of head lice, rosacea associated with Demodex mites (U.S. Pat. No. 5,952,372), and acne rosacea (U.S. Pat. No. 6,133,310) and acne vulgaris (U.S. Pat. No. 6,399,652 B1).

An earlier study noted that when patients suffering from onchoceriasis were treated with ivermectin, the episodes of epileptic attack typically featured in onchoceriasis, reduced (Ndahura, M., et al. (2019). PO 8576 Effect of ivermectin treatment on the frequency of seizures in persons with onchocerciasis-associated epilepsy: preliminary results of a randomized clinical trial. BMJ Global Health, 4(Suppl 3). doi: 10.1136/bmjgh-2019-edc.150). This may be attributed to the abatement of the underlying parasitic disease.

The currently used medications for neurological disorders featuring seizure or movement disorders show significant side effect such as behavioral changes, lethargy, insomnia, clinical depression, psychotic behavior, respiratory depression, coma and death, particularly when taken in overdoses or for long periods of time. Thus, there is an unmet need for development of alterative drugs that can be used to treat such neurological disorders that cause little or no adverse health risks.

### SUMMARY OF THE INVENTION

Ivermectin, and other avermectins, are disclosed herein for the treatment of various neurological disorders associated with GABAergic or glycinergic dysfunction, such as schizophrenia, autism, Parkinson's disease, Alzheimer, stiff-person syndrome (SPS), Hyperekplexia (startle disease) and particularly neurological diseases resulting in seizure disorder such as epilepsy, Lenox Gastaut syndrome and movement disorders such as dystonia, multiple sclerosis, multiple forms of spasticity including spasticity associated with spinal cord injury, spasticity associated with other diseases including parasitic paresis, spasticity associated with cerebral palsy, incontinence after spinal cord injury in humans without causing significant side effects. Ivermectin may also be used for treatment of neurological disorders caused by an infection such as meningitis, meningoencephalitis, encephalitis, cerebral malaria, Zica infection or abscesses in the central nervous system. Ivermectin, and other avermectins, provide for effective treatment of the neurological disorders, while minimizing the undesirable side effects often associated with conventional therapies.

The present disclosure provides compositions and methods for treating neurological diseases and disorders by administering compositions comprising an avermectin to a patient. The avermectin may be ivermectin or an ivermectin derivative.

The present disclosure provides compositions comprising an avermectin, and particularly ivermectin or an ivermectin derivative, and methods for administration to treat humans suffering from GABAergic dysfunction associated neurological disorders.

The present disclosure provides compositions comprising an avermectin, and particularly ivermectin or an ivermectin derivative, and methods for administration to treat humans suffering from glycinergic dysfunction associated neurological disorders.

The present disclosure provides compositions comprising an avermectin, and particularly ivermectin or an ivermectin, and methods for administration to treat humans suffering from a neurological disorders featuring seizure or movement disorders.

The present disclosure provides compositions comprising an avermectin, and particularly ivermectin or an ivermectin derivative, and methods for administration to treat humans suffering from spasticity resulting from a neurological disorder.

The present disclosure provides compositions comprising an avermectin, and particularly ivermectin or an ivermectin derivative, and methods for administration to treat humans suffering from neurological disorders related to dopaminergic dysregulation.

The present disclosure provides compositions comprising an avermectin, and particularly ivermectin or an ivermectin derivative, and methods for administration to treat humans suffering from a neurological disorders caused by cerebral or spinal cord injury.

The present disclosure provides compositions comprising an avermectin, and particularly ivermectin or an ivermectin derivative, and methods for administration to treat humans suffering from a neurological disorders caused by an infection.

The present disclosure provides compositions comprising an avermectin, and particularly ivermectin or an ivermectin derivative, and methods for administration to treat humans suffering from a refractory neurological disorder.

The present disclosure provides ivermectin compositions and dosing for adjunct or stand-alone treatment of neurological disorders featuring seizure or movement disorders, and particularly epilepsy. For example, the compositions and methods provided herein may allow for the reduction in the amount of additional anti-epilepsy medication administered to the patient, or in other instances may replace one or more anti-epilepsy medication administered to the patient. This decreasing of the need for additional medication to manage the disorder reduces the side effects associated with the long-term intake of the conventional therapies.

Also provided are compositions of an avermectin, and particularly ivermectin or an ivermectin derivative, that are specifically formulated for adult or pediatric use. In a specific aspect, the disclosure provides formulations of ivermectin that mask its bitter taste, thus enhancing the palatability for patients and in particular pediatric patients.

The present disclosure provides compositions of an avermectin, and particularly ivermectin or an ivermectin derivative that are specifically formulated as a liquid-based formulation for oral administration. The liquid-based formulation may be in the form of an emulsion, suspension, solution, elixirs, or syrup in which the avermectin is dissolved and/or suspended, or in the form of a liquid-containing capsule in which the avermectin is dissolved and/or suspended in the liquid portion of the capsule core.

The present disclosure provides compositions of an avermectin, and particularly ivermectin or an ivermectin derivative, that are specifically formulated for extended release.

Also provided are dosing regimens of ivermectin or an ivermectin derivative compositions that are designed for pediatric or adult use. The preferred dose of ivermectin composition to treat a pediatric patient is from about 5 mg/day to about 40 mg/day. The preferred dose of ivermectin composition to treat an adult patient is from about 10 mg/day to about 100 mg/day. In a specific aspect, the dosage schedule is once-a-day for about 30 days, for about 60 days, for about 90 days, or continuously. In a specific aspect, the dosage schedule is daily, 2-4 times a week, 3-5 times a week, weekly, biweekly, monthly, bimonthly or annually for about 90 days, for about 6 months, for about 1 year, or continuously.

### DETAILED DESCRIPTION OF THE INVENTION

The present disclosure provides compositions and methods for treating neurological diseases and disorders. The compositions generally include a compound of C-076 family, i.e. avermectins. More particularly, the disclosure provides a method of prevention, treatment and control of various neurological disorders associated with GABAergic dysfunction, (for e.g. schizophrenia, autism, Parkinson's disease, Alzheimer) or glycinergic dysfunction (for e.g. SPS, startle disease), and particularly neurological diseases resulting in seizure disorders (i.e. epilepsy, Lenox Gastaut syndrome) and movement disorders (i.e. dystonia, multiple sclerosis, multiple forms of spasticity including spasticity associated with spinal cord injury, spasticity associated with other diseases including parasitic paresis, spasticity associated with cerebral palsy, incontinence after spinal cord injury) in humans using a composition comprising an avermectin, and particularly ivermectin or an ivermectin derivative, formulations of the said composition, dosages, and treatment schedules. The disclosure also provides a method of prevention, treatment and control of various neurological disorders caused by an infection (i.e. meningitis, meningoencephalitis, encephalitis, cerebral malaria, Zica infection or abscesses in the central nervous system), using a composition comprising an avermectin, and particularly ivermectin or an ivermectin derivative, formulations of the said composition, dosages, and treatment schedules.

In one aspect, the disclosure provide a method of identifying a patient population who will be most benefitted from the method of prevention, treatment and control disclosed herein.

### Avermectins

Avermectins are a family of four closely related major components, A1a, A2a, B1a and B2a and four minor components A1b, A2b, B1b, B2b which are lower homologs of the corresponding major components. Eight different avermectins were isolated in four pairs of homologue compounds, with a major and minor component usually in ratios of 80:20 to 90:10. Anthelmintics derived from the avermectins include ivermectin, selamectin, doramectin, eprinomectin, moxidectin, and abamectin. The family members show anthelmintic and insecticidal/acaricidal activity in different degree of potencies.

The avermectin or derivative may be compound according to the formula I:

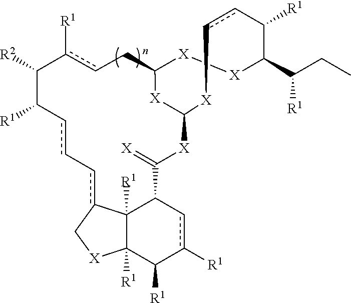

wherein

each R¹ independently is selected from H; OC₁₋₆ saturated or unsaturated alkyl, cycloalkyl, or cycloheteroalkyl; C₁₋₆ saturated or unsaturated alkyl, cycloalkyl, or cycloheteroalkyl; Cl; Br; F; I; OH; OAc; CF₃; NH2; CM; CO₂H; CO₂C₁₋₆ saturated or unsaturated alkyl; NHC₁₋₆ saturated or unsaturated alkyl or cycloalkyl; or N(C₁₋₆ saturated or unsaturated alkyl or cycloalkyl)₂; aryl or heteroaryl;

R² is selected from mono, di, or triglycoside, or OC(O) C₃₋₅ alkenyl;

each X is independently selected from CH₂, N, O, S, SO, or SO₂; and

wherein n=0-6.

The preferred avermectin compounds for the purpose of the present invention include ivermectin or an ivermectin derivative. Ivermectin typically comprises between 70-90% of 22,23-dihydroavermectin Bla and less than about 30% of 22, 23-dihydroavermectin B1b.

Derivatives of ivermectin including abamectin and doramectin, and prodrugs of ivermectin, and have properties and uses similar to ivermectin in use. Abamectin and Doramectin both has a double bond at position C22-C23 in the structural formula of ivermectin. Additionally, in doramectin, position C25 is substituted at the side chain of a benzene ring.

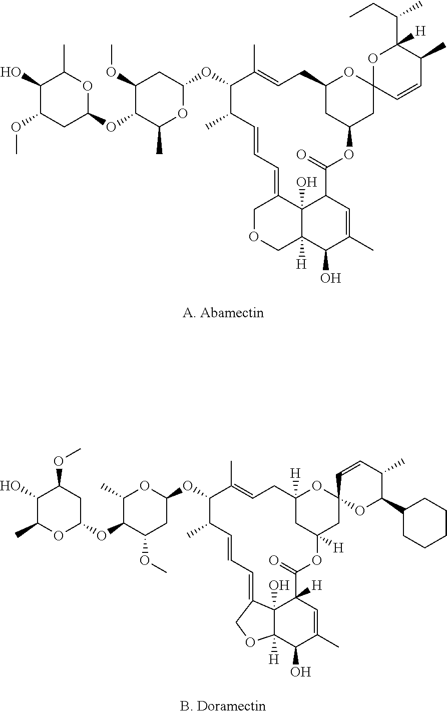 [SMILES: CC[C@H](C)[C@H]1OC2(C=C[C@@H]1C)C[C@@H]1C[C@@H](C/C=C(\C)[C@@H](O[C@H]3C[C@H](OC)[C@@H](O[C@H]4C[C@H](OC)[C@@H](O)C(C)O4)[C@H](C)O3)[C@@H](C)/C=C/C=C3\COC[C@@H]4[C@H](O)C(C)=CC(C(=O)O1)[C@]34O)O2 ; CO[C@H]1C[C@H](O[C@H]2[C@H](C)O[C@@H](O[C@@H]3/C(C)=C/C[C@@H]4C[C@@H](C[C@]5(C=C[C@H](C)[C@@H](C6CCCCC6)O5)O4)OC(=O)[C@@H]4C=C(C)[C@@H](O)[C@H]5OC/C(=C\C=C\[C@@H]3C)[C@]54O)C[C@@H]2OC)O[C@@H](C)[C@@H]1O]

As used herein, “derivative” to a compound that retains the biological activity of the parent avermectin from which it is derived, or is a prodrug for the parent avermectin. Derivatives may include esters, amides, ethers or the like that are derived from the avermectin.

### Methods of Treatment

In one aspect, the disclosure provides a method of treating, preventing and/or reducing the severity or extent of neurological disorders featuring seizure or movement disorders by administering to a subject in need thereof a therapeutically effective amount of a composition, or compositions, comprising one or more avermectin, and particularly ivermectin or an ivermectin derivative.

As used herein, “treating”, “treat”, “treatment” refer to reducing, relieving, ameliorating, or alleviating at least one of the symptoms of the disease or disorder.

The terms “prevent”, “prevention”, and the like refer to acting prior to overt disease or disorder onset, to prevent the disease or disorder from developing or to minimize the extent of the disease or disorder, or slow its course of development.

The term “cure” and the like means to heal, to make well, or to restore to good health or to allow a time without recurrence of disease so that the risk of recurrence is small.

The phrase “therapeutically effective amount” is used herein to mean an amount sufficient to results in a desired beneficial change of physiology in the subject or to cause an improvement in a clinically significant condition in the subject, for example, by delaying, reducing, minimizing or mitigating one or more symptoms associated with the disease or disorder.

The subjects receiving the therapy described herein (e.g. a therapeutically effective amount of a composition, or compositions, comprising one or more ivermectin or ivermectin derivatives) may experience as a result of the therapy a reduction or complete absence of seizures and/or reduction/absence of involuntary muscle movements due to a disorder.

### Neurological Diseases

In one aspect, the disclosure provides a method for treating, preventing and/or reducing the severity or extent of neurological disorders featuring a seizure disorder including but not limited to epilepsy, treatment-resistant epilepsy, Gravet syndrome, Lenox Gastaut syndrome, spinal cord injury, childhood absence 5 (ECA5), epileptic encephalopathy (EE), early infantile epileptic encephalopathy 43 (EIEE43), Angelman syndrome, injury to brain, stroke, addictive behavior, subarachnoid hemorrhage, anoxic encephalopathy, infectious or metabolic encephalopathy, hemorrhagic, embolic/athersclerotic cerebrovascular accidents, and other seizure indications.

In another aspect, the disclosure provides a method for treating, preventing and/or reducing the severity or extent of neurological disorders associated with muscle movement disorders including but not limited to multiple sclerosis, multiple forms of spasticity including spasticity associated with spinal cord injury, spasticity associated with other diseases including parasitic paresis, spasticity associated with cerebral palsy, incontinence after spinal cord injury, dystonia, lateral sclerosis, myotonic dystrophy, congenital (hereditary) muscular dystrophies, e.g. Duchenne's and Becker's, Rett syndrome, Prader-Willi syndrome and any orphan motor neuron diseases.

In one aspect, the disclosure provides a method for treating, preventing and/or reducing the severity or extent of neurological disorders caused by GABAergic dysfunction including but not limited to Alzheimer's, Parkinson's disease, Schizophrenia, Autism, autism spectrum disorder, global developmental delay, decreased fine and gross motor control, attention deficit hyperactivity disorder (ADHD).

In one aspect, the disclosure provides a method for treating, preventing and/or reducing the severity or extent of neurological disorders caused by glycinergic dysfunction including but not limited to stiff person syndrome, startle disease.

In one aspect, the disclosure provides a method to treating, preventing and/or reducing the severity or extent of neurological disorders caused by an infection including but not limited to mycobacterium infection, Zica infection, and cerebral malaria.

In one aspect, the disclosure provides a method to treating, preventing and/or reducing the severity or extent of neurological disorders caused by an injury.

### Pharmaceutical Composition/Formulation

The pharmaceutical composition comprises one or more avermectin, and particularly comprises ivermectin or ivermectin derivatives. These compositions may be administered alone or in combination with other agents for the treatment of the disorders and diseases disclosed herein.

A pharmaceutical composition may refer to a composition comprising a therapeutically effective compound and a pharmaceutically acceptable carrier and optionally, other materials, e.g., one or more inert components (for example, a detectable agent or label) or one or more active components.

The term “carrier” refers to a diluent, adjuvant, excipient, or vehicle in which the pharmaceutical composition is administered. Pharmaceutically acceptable carriers may include one or more solvents, dispersion media, coatings, isotonic and absorption delaying agents, and the like that are physiologically compatible. Compositions can include components such as diluents, binders, stabilizers, buffers, salts, lipophilic solvents, preservatives, or mixtures thereof. Examples of pharmaceutically acceptable carriers include but are not limited to water, saline, phosphate buffered saline, aqueous dextrose solutions, glyceral solutions. Suitable pharmaceutical excipients include starch, glucose, lactose, sucrose, dextrose, gelatin, mannitol, cellulose malt, rice, flour, chalk, silica gel, sodium stearate, glycerol monostearate, talc, sodium chloride, milk powder, glycerol, propylene, glycol, water, ethanol and the like.

Carriers may also encompass a buffer or pH adjusting agent such as a salt prepared from an organic acid or base optionally mixed with a nontoxic surfactant. Examples of buffers include but are not limited to organic acid salts such as salts of citric acid, ascorbic acid, gluconic acid, carbonic acid, tartaric acid, succinic acid, acetic acid, or phthalic acid, Tris, tromethamine hydrochloride, and phosphate buffers. Additional carriers may include polymeric excipients or additives such as polyvinylpyrrolidones, ficolls (a polymeric sugar), dextrates (e.g., cyclodextrins, such as 2-hydroxypropyl-.quadrature.-cyclodextrin), polyethylene glycols, flavoring agents such as cherry or wintergreen flavor, antimicrobial agents, sweeteners, antioxidants, antistatic agents. The compositions may include a pharmaceutical carrier or excipient and an avermectin as the/an active agent, and, in addition, may include other medicinal agents, pharmaceutical agents, carriers, adjuvants, binding agents etc. A pharmaceutical composition of the invention may also contain minor amounts of auxiliary substances such as wetting or emulsifying agents, pH buffering agents, antioxidants, and the like, such as, for example, citric acid, sorbitan monolaurate, triethanolamine oleate, butylalted hydroxytoluene, etc.

The compositions can take the form of solid, semi-solid, lyophilized powder, or liquid dosage forms, such as for example, solutions, suspensions, emulsion, aerosols, gels, implants, microneedles, tablets, pills, capsules, soft elastic or hard gelatin capsules, dermal patch, gummy bears, powders, suspensions, extended-release formulations and the like preferably in unit dosage forms suitable for simple administration of precise dosages. In a preferred embodiment the composition takes the forms of tablets, capsules, liquid caps, sublingual dissolving tablets, sublingual spray, nasal spray, gummy bear and/or dermal patch.

In a preferred aspect, the composition is a liquid-based formulation including but not limited to an emulsion, suspension, solution, elixirs, or syrup in which the avermectin is dissolved and/or suspended, or in the form of a liquid-containing capsule in which the avermectin is dissolved and/or suspended in the liquid portion of the capsule core. The composition may be a capsule filled with an effective therapeutic amount of the liquid pharmaceutical formulation. In other aspects, the dosage is a solid oral dosage form (i.e., tablet, capsule, etc.) comprising the avermectin wherein the formulation is released in the patient's body in as an extended release and/or delayed release.

### Method of Administration

The method of administrating the composition comprising ivermectin or ivermectin derivative in an appropriate pharmaceutical composition can be carried out via oral, nasal, intraocular, intravenous, intramuscular, subcutaneous, transdermal, subdermal, sublingual or rectal administration.

In one aspect, the route of administration is oral using a dosage regimen that can be adjusted according to the degree of severity of the disease-state to be treated in the form of a tablet, sublingual dissolving tablets, pills, capsules, soft elastic or hard gelatin capsules, gummy bears and/or sublingual dissolving spray.

In one aspect, the route of administration is nasal using a dosage regimen that can be adjusted according to the degree of severity of the disease-state to be treated. The composition can be an aerosol, a liquid suspension, a liquid dispersion, a powder, or an aqueous solution based formulation in the form of a nasal spray, a nasal douche, an inhaler, a nasal drop, and/or a diffuser.

In one aspect, the route of administration is dermal including but not limited to topical, subcutaneous, subdermal, transdermal, intradermal or dermal patch.

An effective amount of the composition may be co-administered by two different routes of administration, for example, by oral administration and by dermal administration. Co-administration can be performed at approximately the same time, or at different times. An effective amount of the composition may be co-administered with other medications commonly in use for treating neurological disorders.

### Patient Population

The patient is may be a human or other mammal. In human patients are typically having a neurological disorder featuring a seizure disorder and/or a movement disorder.

In some embodiments, the patient has a refractory neurological disorder. The term “refractory” is used herein means diseases that do not respond, or have reduced response, to one or more treatments used for that particular disease. The disease may be unresponsive at the beginning of treatment, or becomes nonresponsive during treatment.

In some embodiments, the patient has a neurological disorder associated with an infection or injury.

In some embodiments, the patient has a neurological disorder associated with GABAergic dysfunction.

In some embodiments, the patient has a neurological disorder associated with glycinergic dysfunction.

### Dosage

An effective amount of the composition is an amount sufficient to confer a therapeutic benefit in a patient after administration for example to improve in the subject one or more symptoms of the disease. The effective amount may vary depending on the species, age, weight, health of the subject and the nature or severity of the disease. Depending on the mode of administration, the effective amount may vary as well. In some cases, multiple doses of the composition are administered to achieve the effective amount for the therapeutic benefit intended. In some embodiments, the dose of avermectin, and particularly ivermectin or ivermectin derivative, administered is about 1 mg/day to about 150 mg/day, about 5 mg/day to about 150 mg/day, about 10 mg/day to about 150 mg/day, about 20 mg/day to about 150 mg/day, about 25 mg/day to about 150 mg/day, about 30 mg/day to about 150 mg/day, about 35 mg/day to about 150 mg/day, about 40 mg/day to about 150 mg/day, about 50 mg/day to about 150 mg/day, about 60 mg/day to about 150 mg/day, about 70 mg/day to about 150 mg/day, about 80 mg/day to about 150 mg/day, about 90 mg/day to about 150 mg/day, about 100 mg/day to about 150 mg/day, about 110 mg/day to about 150 mg/day, about 120 mg/day to about 150 mg/day, about 130 mg/day to about 150 mg/day about 140 mg/day to about 150 mg/day, about 150 mg/day. In a preferred embodiment, the dose of ivermectin or ivermectin derivatives administered to a pediatric patient is about 5 mg/day to about 40 mg/day. In a preferred embodiment, the dose of ivermectin or ivermectin derivative administered to a pediatric patient is about 10 mg/day to about 100 mg/day.

The dosage regimens for the therapy may be adjusted to provide the optimum desired response (e.g., a therapeutic or prophylactic response). For example, a single dose may be administered, several divided doses may be administered over time or the dose may be proportionally reduced or increased depending on the subject's responsiveness to the therapy. In some embodiments, the dosage schedule is once a day for about 30 days, for about 60 days, for about 90 days, or continuously. In some embodiments, the dosage schedule is daily, 2-4 times a week, 3-5 times a week, weekly, biweekly, monthly, or bimonthly for about 90 days, for about 6 months, for about 1 year or continuously.

Kits of present invention can include any combination of agents, compositions, components, reagents, administration devices or mechanisms, or other entities provided herein. For instance, a kit of the present invention may include one or more ivermectin or ivermectin derivatives and one or more of a carrier composition, an administration device, and a combination therapy agent. Kits may further include a device to facilitate delivery. Any of the kits provided herein can be included in a container, pack, or dispenser together with instructions for administration.

We claim:

1. A method for treating a neurological disorder comprising administering a composition comprising an effective amount of an avermectin to a subject in need of such treatment.

2. The method of claim 1, wherein the composition comprises a compound of formula I: 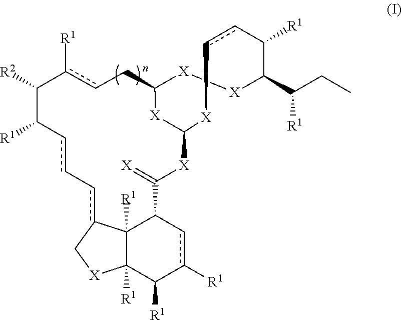
wherein
each le independently is selected from H; OC₁₋₆ saturated or unsaturated alkyl, cycloalkyl, or cycloheteroalkyl; C₁₋₆ saturated or unsaturated alkyl, cycloalkyl, or cycloheteroalkyl; Cl; Br; F; I; OH; OAc; CF₃; NH₂; CM; CO₂H; CO₂C₁₋₆ saturated or unsaturated alkyl; NHC₁₋₆ saturated or unsaturated alkyl, or cycloalkyl; N(C₁₋₆ saturated or unsaturated alkyl, or cycloalkyl)₂; aryl and heteroaryl;
R² is selected from mono, di, or triglycoside, or OC(O)C₃₋₅ alkenyl;
each X is independently selected from CH₂, N, O, S, SO, or SO₂; and
wherein n=0-6;
or a pharmaceutically acceptable salt thereof, or a combination thereof.

3. The method of claim 1 or 2, wherein the avermectin is an ivermectin or an ivermectin derivative.

4. The method of claim 1, 2, or 3 where the subject is a mammal.

5. The method of claim 4 where the subject is a human.

6. The method of any one of claims 1-5 where the neurological disorder is characterized by seizures and/or movement disorders.

7. The method of any one of claims 1-6 where the neurological disorder features seizure disorder.

8. The method of any one of claims 1-7 where the neurological disorder features a movement disorder.

9. The method of any one of claims 1-8 where the neurological disorder features spasticity.

10. The method of any one of claims 1-9 where the neurological disorder is caused by GABAergic or glycinergic dysfunction.

11. The method of any one of claims 1-10 where the neurological disorder is caused by GABAergic dysfunction.

12. The method of any one of claims 1-10 where the neurological disorder is caused by glycinergic dysfunction.

13. The method of any one of claims 1-9 where the neurological disorder is caused by an injury to the nervous system.

14. The method of any one of claims 1-10 or 13 where the neurological disorder is caused by an injury to brain.

15. The method of claim any one of claims 1-10 or 13 where the neurological disorder is caused by an injury to spinal cord.

16. The method of any one of claims 1-10 where the neurological disorder is caused by a parasite.

17. The method of any one of claims 1-10 or 16 where the neurological disorder is caused by an infection.

18. The method of any one of claims 1-10 or 16-17 where the infection is bacterial.

19. The method of any one of claims 1-10 or 16-17 where the infection is viral.

20. The method of any one of claims 1-19 where the neurological disorder is refractory.

21. The method of any one of claims 1-20 where the neurological disorder selected from epilepsy, refractory epilepsy, Gravet syndrome, Lenox Gastaut syndrome, spinal cord injury, childhood absence 5 (ECA5), epileptic encephalopathy (EE), early infantile epileptic encephalopathy 43 (EIEE43), Angelman syndrome, injury to brain, stroke, addictive behavior, subarachnoid hemorrhage, anoxic encephalopathy, infectious or metabolic encephalopathy, hemorrhagic, embolic/athersclerotic cerebrovascular accidents.

22. The method of any one of claims 1-21 where the neurological disorder is epilepsy.

23. The method of any one of claims 1-22 where the neurological disorder is refractory epilepsy.

24. The method of any one of claims 1-20 where the neurological disorder is selected from multiple sclerosis, multiple forms of spasticity including spasticity associated with spinal cord injury, spasticity associated with other diseases including parasitic paresis, spasticity associated with cerebral palsy, incontinence after spinal cord injury, dystonia, lateral sclerosis, myotonic dystrophy, congenital (hereditary) muscular dystrophies, e.g. Duchenne's and Becker's, Rett syndrome, Prader-Willi syndrome.

25. The method of any one of claims 1-20 where the neurological disorder is selected from Alzheimer's, Parkinson's disease, Schizophrenia, Autism, autism spectrum disorder, global developmental delay, decreased fine and gross motor control, attention deficit hyperactivity disorder (ADHD).

26. The method of any one of claims 1-20 where the neurological disorder is selected from startle disease, stiff person syndrome.

27. The method of any one of claims 1-26 where the neurological disorder is selected from a mycobacterium infection, Zica infection, and cerebral malaria.

28. The method of any one of claims 1-27, wherein the composition is used in combination with another agent.

29. The method of any one of claims 1-28 where the route of administration is oral, nasal, ocular, intravenous, subdermal, transdermal, subcutaneous injection, topical, or rectal.

30. The method of any one of claims 1-29 where the composition is administered at doses between 1 mg/day to 150 mg/day.

31. The method of any one of claims 1-30 where the dose is between 1 mg/day to 40 mg/day, or between 5 mg/day to 40 mg/day, or between 10 mg/day to 40 mg/day, or between 10 mg/day to 100 mg/day, or between 10 mg/day to 150 mg/day.

32. The method of any one of claims 1-31 where the dosage is daily, or 2-4 times per week, or 3-5 times a week, or weekly, or biweekly, or monthly, or bimonthly, for 90 days, or for 6 months, or for 1 year, or is continuous.

33. A composition comprising a therapeutically effective amount of an avermectin, or a compound of Formula I, or ivermectin, or a pharmaceutically acceptable salt or stereoisomer thereof.

34. A formulation comprising a therapeutically effective amount of an avermectin, or a compound of Formula I, or ivermectin, or a pharmaceutically acceptable salt or stereoisomer thereof, together with one or more materials selected from the group consisting of excipients, carriers, fillers, extenders, binders, humectants, disintegrating agents, solution retarders, absorption accelerators, wetting agents, adsorbents, lubricants, and buffering agent.

35. The formulation of claim 33 or 34 where the formulation is a solid dosage form.

36. The formulation of any one of claim 33-35 where the solid dosage form is a pill, tablet, liquid caps, sublingual dissolving tablets, gummy bear or capsule.

37. The formulation of claim 33 or 34 where the formulation is a liquid dosage form.

38. The formulation of any one of claim 33-34 or 37 where the liquid dosage form is a emulsion, suspension, solution, elixirs, syrup or a liquid containing capsule.

39. The formulation of claim 33 or 34 where the formulation is an aerosol.

40. The formulation of any one of claim 33-34 or 40 where the formulation is in the form of sublingual spray or nasal spray.
